(19) United States
(12) Patent Application Publication
(10) Pub. No.: US 2025/0204888 A1
(43) Pub. Date: Jun. 26, 2025

(54) METHODS AND SYSTEMS FOR ACQUISITION OF PATIENT INFORMATION FOR ULTRASOUND SCANS VIA BARCODE EXTRACTION

(71) Applicant: BFLY OPERATIONS, INC., Burlington, MA (US)

(72) Inventors: Mark Olschesky, Medford, MA (US); Maureen Dai, Guilford, CT (US); Brandon Fiegoli, New York, NY (US); Katherine Karg, Burlington, MA (US)

(21) Appl. No.: 18/852,007

(22) PCT Filed: Jan. 31, 2023
(86) PCT No.: PCT/US2023/011946

§ 371 Date: Sep. 27, 2024

Related U.S. Application Data
(60) Provisional application No. 63/326,245, filed on Mar. 31, 2022.

(51) Int. Cl. A61B 8/00; G16H 10/60; G16H 40/63

(52) U.S. Cl. CPC A61B 8/4438; A61B 8/565; G16H 10/60; G16H 40/63

(57) ABSTRACT

Described herein are methods and systems for acquisition of patient information for ultrasound scans via barcode extraction. An ultrasound system may include an ultrasound device, a mobile device in operative communication with the ultrasound device, a processing device, and a cloud including one or more servers. The processing device may be configured to prompt for and receive selection of a barcode type, and prompt for and receive configuration of how to process barcode data, where the prompt is based on the selected barcode type The mobile device may be configured to download barcode settings from the cloud, perform an ultrasound scan on a patient in conjunction with the ultrasound device, scan a barcode associated with the patient, process the barcode data based on the downloaded barcode settings, and perform an electronic health record query based on data from the processed barcode.

### CROSS-REFERENCE TO RELATED APPLICATIONS

This application claims the benefit of and priority to U.S. Provisional Patent Application No. 63/326,245 filed Mar. 31, 2022. The entire disclosure of the foregoing application is incorporated by referenced herein.

### FIELD

Generally, the aspects of the technology described herein relate to ultrasound scanning. Certain aspects relate to acquisition of patient information for ultrasound scans via barcode extraction.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the present technology, an ultrasound system includes an ultrasound device, a mobile device in operative communication with the ultrasound device and running an ultrasound application, a processing device, and a cloud including one or more servers, where the ultrasound device and the processing device are in communication with the cloud. The processing device is configured to prompt for and receive a selection of a barcode type. The processing device is further configured to prompt for and receive configuration of how to process barcode data, where the prompt is based on the selected barcode type. The mobile device is configured to download from the cloud barcode settings including the barcode type and the configuration of how to process the barcode data, perform an ultrasound scan on a patient in conjunction with the ultrasound device, scan a barcode associated with the patient, process the barcode data based on the downloaded barcode settings, and perform an electronic health record (EHR) query based on data from the processed barcode.

In some embodiments, the processing device is configured, when prompting for and receiving the selection of the barcode type, to prompt for and receive a selection of a 1D non-GSI barcode, a 1D GS1 barcode, or a 2D barcode. In some embodiments, the processing device is configured, when prompting for and receiving the configuration of how to process the barcode data, to prompt for a selection of a type of data encoded by the barcode when the selected barcode type is a 1D non-GSI barcode. In some embodiments, the processing device is configured, when prompting for and receiving the configuration of how to process the barcode data, to prompt for input of application identifiers and a data type associated with each of the application identifiers when the selected barcode type is a 1D GSI barcode. In some embodiments, the processing device is configured, when prompting for and receiving the configuration of how to process the barcode data, to prompt for selection of a certain number of delimiters and a data type associated with each of the delimiters. In some embodiments, the data type includes a first name, last name, date of birth, gender/sex, patient ID, encounter ID, or blank.

In some embodiments, the processing device is further configured to determine that automatic EHR querying should be presented as an option. In some embodiments, the processing device is configured to determine that automatic EHR querying should be presented as an option based on querying using Fast Healthcare Interoperability Resources being enabled. In some embodiments, the processing device is further configured to prompt for and receive a selection of whether to automatically perform the EHR query based on the data from the processed barcode after the ultrasound scan. In some embodiments, the barcode settings further include whether to automatically perform the EHR query based on the data from the processed barcode after the ultrasound scan.

In some embodiments, the barcode settings are associated with a particular department, and the mobile device is further configured, when downloading the barcode settings from the cloud, to check whether new department-specific settings are available from the cloud when running the ultrasound application under an account linked to the particular department. In some embodiments, the mobile device is configured, when scanning the barcode associated with the patient, to scan a barcode printed on a bracelet worn by the patient. In some embodiments, the mobile device includes a camera and is configured, when scanning the barcode associated with the patient, to capture an image of the barcode using the camera. In some embodiments, the mobile device is configured, when performing the EHR query based on the data from the processed barcode, to query the EHR for active encounters associated with a patient ID when the barcode contains the patient ID. In some embodiments, the mobile device is configured, when performing the EHR query based on the data from the processed barcode, to query the EHR for active encounters associated with an encounter ID when the barcode contains the encounter ID. In some embodiments, the mobile device is further configured to return a list of matching encounters from the EHR query, prompt for selection of one of the matching encounters, and associate the ultrasound scan with patient information from the selected encounter. In some embodiments, the mobile device is further configured, when there is only one matching encounter from the EHR query, to automatically associate the ultrasound scan with patient information from the matching encounter. In some embodiments, the processing device is further configured to prompt for and receive a selection as to whether the mobile device should automatically associate the ultrasound scan with patient information if there is only one matching encounter from the EHR query. In some embodiments, the processing device is further configured to prompt for and receive a selection as to whether the mobile device should automatically start every patient association with barcode scanning.

Some aspects include a method of performing the above aspects and embodiments. Some aspects include at least one non-transitory computer-readable storage medium on a processing device storing processor-executable instructions that, when executed by at least one processor, cause the at least one processor to perform the above aspects and embodiments of a processing device. Some aspects include at least one non-transitory computer-readable storage medium on a mobile device storing processor-executable instructions that, when executed by at least one processor, cause the at least one processor to perform the above aspects and embodiments of a mobile device.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example configuration for ultrasound scanning, in accordance with certain embodiments described herein;

FIG. 2 illustrates a process for selecting barcode scanning settings for a healthcare department's ultrasound application, in accordance with certain embodiments described herein;

FIG. 3 illustrates a process for scanning a patient barcode using an ultrasound application running on a mobile device, in accordance with certain embodiments described herein.

FIG. 4 illustrates an example graphical user interface (GUI) that may be displayed by a processing device, in accordance with certain embodiments described herein;

FIG. 5 illustrates an example GUI that may be displayed by a processing device, in accordance with certain embodiments described herein;

FIG. 6 illustrates an example GUI that may be displayed by a processing device, in accordance with certain embodiments described herein;

FIG. 7 illustrates another example of the GUI of FIG. 6, in accordance with certain embodiments described herein;

FIG. 8 illustrates an example GUI that may be displayed by a processing device in accordance with certain embodiments described herein;

FIG. 9 illustrates another example of the GUI of FIG. 8, in accordance with certain embodiments described herein;

FIG. 10 illustrates another example of the GUI of FIG. 6, in accordance with certain embodiments described herein;

FIG. 11 illustrates another example of the GUI of FIG. 6, in accordance with certain embodiments described herein;

FIG. 12 illustrates an example GUI that may be displayed by a mobile device, in accordance with certain embodiments described herein;

FIG. 13 illustrates an example GUI that may be displayed by a mobile device, in accordance with certain embodiments described herein; and

FIG. 14 illustrates an example GUI that may be displayed by a mobile device, in accordance with certain embodiments described herein.

### DETAILED DESCRIPTION

Healthcare departments may prefer clinicians such as doctors and nurses to associate patient information with ultrasound scans in a consistent manner. Patient information may include, among other items, the patient ID (a.k.a., the patient medical record number (MRN)) and the encounter ID (a.k.a., the contact serial number (CSN) or visit number). However, associating patient information with a scan can be time-consuming both for the medical professionals to perform and for the department's technology team to set up and maintain. In some situations, enabling association of patient information with scans may require set up of worklists, while in other situations, worklists may not be viable. Additionally, data validation is an essential part of healthcare workflows. When there is an opportunity for a user to freely enter patient information, or a user is asked to select patient information from a long list, this leaves an opportunity for the wrong info to be entered.

The inventors have recognized that barcodes may present an opportunity for simpler association of patient information with ultrasound scans and for validation of patient information. However, the inventors have also recognized that there is non-trivial variability in barcode formats and the content encoded in a barcode. The inventors have developed a flexible and scalable solution to enable the proper extraction of information from certain barcode formats commonly used in medical settings, and to enable rapid validation of the extracted data by querying the department's electronic health records (EHR).

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

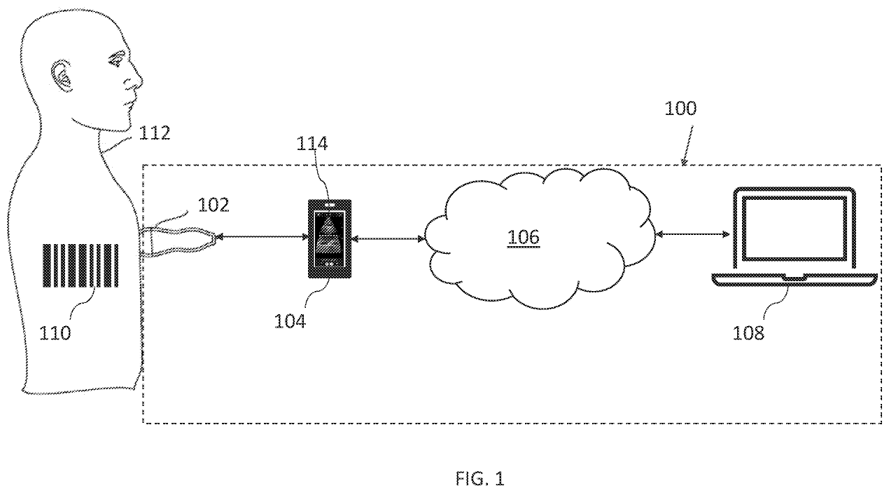

FIG. 1 illustrates an example configuration for ultrasound scanning, in accordance with certain embodiments described herein. FIG. 1 illustrates an ultrasound device 102, a mobile device 104 running an ultrasound application (“app”) 114, the cloud 106, a processing device 108, a barcode 110, and a patient 112. The ultrasound device 102 may be, for example, an ultrasound probe. The mobile device 104 is in operative communication (either through a wireless or wired connection) with the ultrasound device 102 and runs the ultrasound application 114 for interfacing with the ultrasound device 102. For example, the ultrasound application 114 may enable a user to use a graphical user interface (GUI) displayed on the mobile device 104 to configure the ultrasound device 102 for a particular type of ultrasound scan, receive ultrasound data from the ultrasound device 102, display ultrasound images in the GUI, and upload ultrasound images to the cloud 106. The mobile device 104 may be, for example, a smartphone, tablet, or laptop. As illustrated, both the mobile device 104 and the processing device 108 are connected to the cloud 106, for example through wireless networks (e.g., Wi-Fi). The cloud 106 may include, for example, one or more servers and software and databases on those servers which the processing device 108 and the mobile device 104 can access. The mobile device 104 may be one of multiple mobile devices 104 associated with different clinicians such as doctors and nurses in the healthcare department. For example, the mobile devices 104 may be owned by and/or used by doctors and nurses who work for the healthcare department. The ultrasound application 114 running on these mobile devices 104 may be configured with department-specific settings. For example, a clinician may set up an account with the ultrasound application 114 and link the account to the healthcare department. Mobile devices 104 may download the department-specific settings for the ultrasound application 114 from the cloud 106. The ultrasound device 102, the mobile device 104, the ultrasound application 104, the cloud 106, and the processing device 108 may be considered to be an ultrasound system 100, although an ultrasound system may also include fewer or more elements.

The processing device 108 may be associated with a technology administrator for the healthcare department. While the processing device 108 is illustrated as a laptop, any type of processing device may be used as the processing device 108. The administrator may use the processing device 108 to configure department-wide settings for the ultrasound application 114 which may apply when a user has logged into the ultrasound application 114 with an account linked to the department. For example, department-wide settings may include settings related to credentialing, documentation, and billing. Additionally, the administrator may use the processing device 108 to configure department-wide barcode scanning for the ultrasound application 114. The barcode 110 may be associated with the patient, for example by printing the barcode 110 on a bracelet and attaching the bracelet to the patient (not illustrated in FIG. 1). The mobile device 104 may include a camera (not illustrated in FIG. 1), and the ultrasound application 114 running on the mobile device 104 may use the camera to scan the barcode 110 on the patient 112 in conjunction with performing an ultrasound scan with the ultrasound device 102 and mobile device 104 in order to associate the ultrasound scan with the patient 112.

Following is a description of certain barcode formats commonly used in healthcare systems.
1. One-dimensional (1D) non-GSI barcode. Such a barcode may encode a value but lack a mechanism for imparting meaning upon that data. For example, the data encoded by a 1D non-GS1 barcode may be intended to be a patient ID, but the barcode itself may not have a mechanism for imparting to a barcode scanner that the value encoded is a patient ID.
2. 1D GS1 barcode. GS1 is an organization and data standard for managing metadata of barcodes. A GSI barcode embeds application identifiers (AIs). AIs allow the barcode scanner to identify specific fields within a barcode and validate the information in those fields based on defined length/check sums. Each AI may be associated with specific types of patient information. Table 1 provides an example of two AIs each of which is associated with a particular type of patient information:

TABLE 1
Association of application identifiers (AIs) with particular
types of patient information in a 1D GS1 barcode

| | Encoded |
|---|---|
| AI | Information Type |
| 01 | Patient ID |
| 3103 | Encounter ID |

3. Two-dimensional (2D) (a.k.a., Data Matrix) barcode. 2D barcodes may be able to embed thousands of bits of information. 2D barcodes may have content blocks that are delineated and associated with particular types of information using delimiters, and which allow the scanner to extract strings and content and match them to the proper fields. Table 2 provides an example of seven delimiters each of which is associated with a particular type of patient information (aside from one delimiter which is not associated with information, labeled “BLANK”):

TABLE 2
Association of delimiters with particular types
of patient information in a 2D barcode

| | Encoded |
|---|---|
| Delimiter | Information Type |
| 1 | Last Name |
| 2 | First Name |
| 3 | Date of Birth |
| 4 | Gender/Sex |
| 5 | BLANK |
| 6 | Encounter ID |
| 7 | Patient ID |

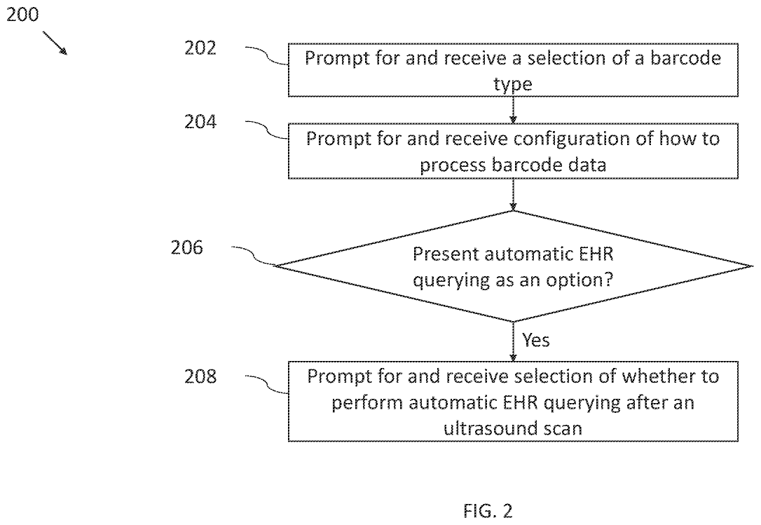

FIG. 2 illustrates a process 200 for selecting barcode scanning settings for a healthcare department's ultrasound application (e.g., the ultrasound application 114), in accordance with certain embodiments described herein. Settings for a healthcare department's ultrasound application may mean settings used for the ultrasound application 114 when a user has logged into an account on the ultrasound application 114, and the account is linked to the department, and the ultrasound application 114 has been configured with particular settings specific to the department. The process 200 may be performed, for example, by the processing device 108. In particular, selection of the settings may occur through internet-based ultrasound software (which may be software as a service (SAAS)) accessed by the processing device from the cloud (e.g., the cloud 106). Once the settings have been selected in the cloud-based environment, configuration of the department's ultrasound application 114 with these settings may occur when mobile devices (e.g., the mobile devices 104) that are running the ultrasound application 114 under an account linked to the department download the settings from the cloud.

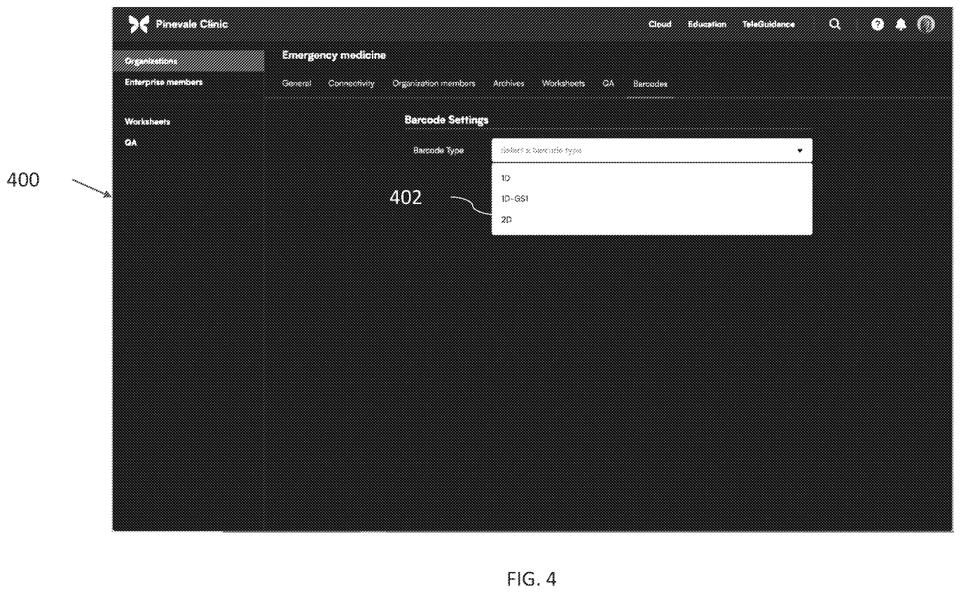

At step 202, the processing device prompts for and receives a selection from a user (e.g., the department technology administrator) of a barcode type. For example, the processing device may prompt for and receive a selection of a 1D non-GSI barcode, a 1D GS1 barcode, or a 2D barcode. FIG. 4 illustrates an example GUI 400 that may be displayed by the processing device (e.g., in conjunction with step 202), in accordance with certain embodiments described herein. The GUI 400 includes a menu 402 from which the user may select a barcode type. In the example GUI 400, the barcode options are “ID” (i.e., 1D non-GSI barcode), “ID-GS1” (i.e., 1D GS1 barcode), and “2D” (i.e., a 2D barcode). The process 200 proceeds to step 204.

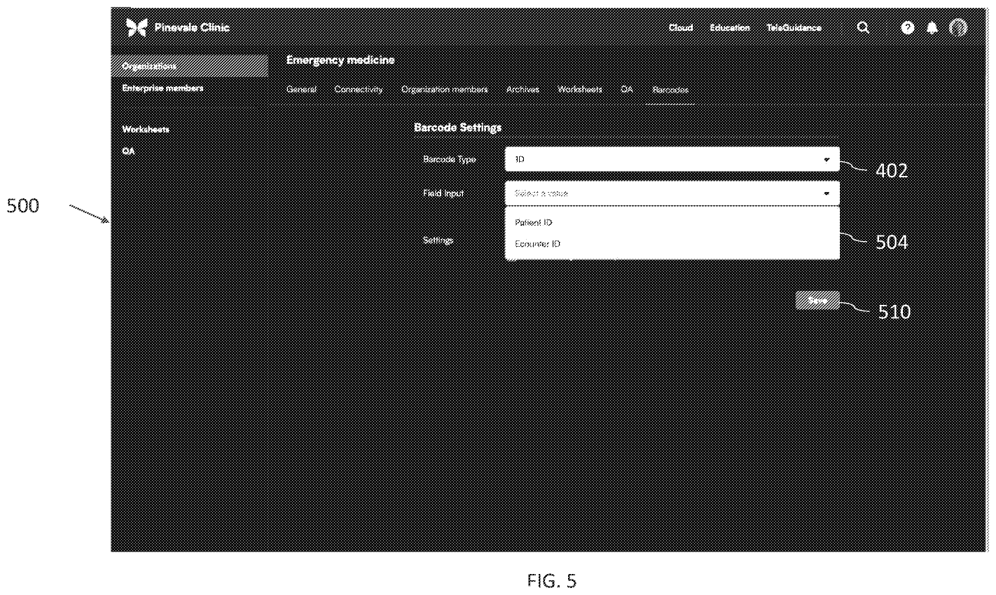

At step 204, the processing device prompts for and receives configuration of how to process barcode data, where the prompt is based on the barcode type selected at step 202. For example, if a 1D non-GSI barcode type is selected at step 202, then the processing device may prompt the user for a selection of the data type encoded by the barcode. FIG. 5 illustrates an example GUI 500 that may be displayed by the processing device (e.g., in conjunction with step 204), in accordance with certain embodiments described herein. The GUI 500 displays barcode data processing settings specific for selection of a 1D non-GSI barcode type (as selected from the menu 402). The GUI 500 includes a menu 504 from which the user may select one type of data encoded by the barcode. In the example GUI 500, the data type options are patient ID and encounter ID.

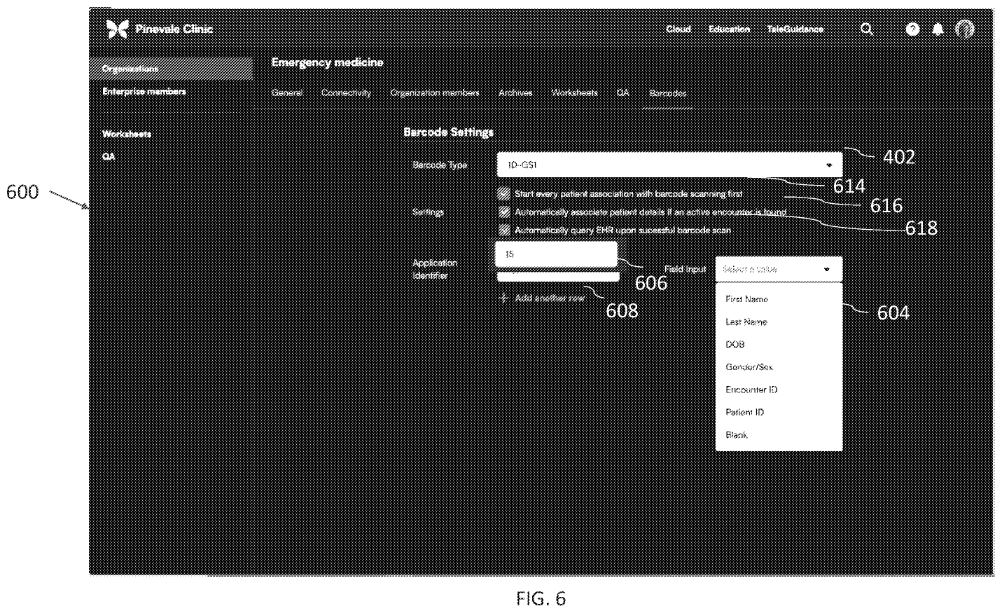

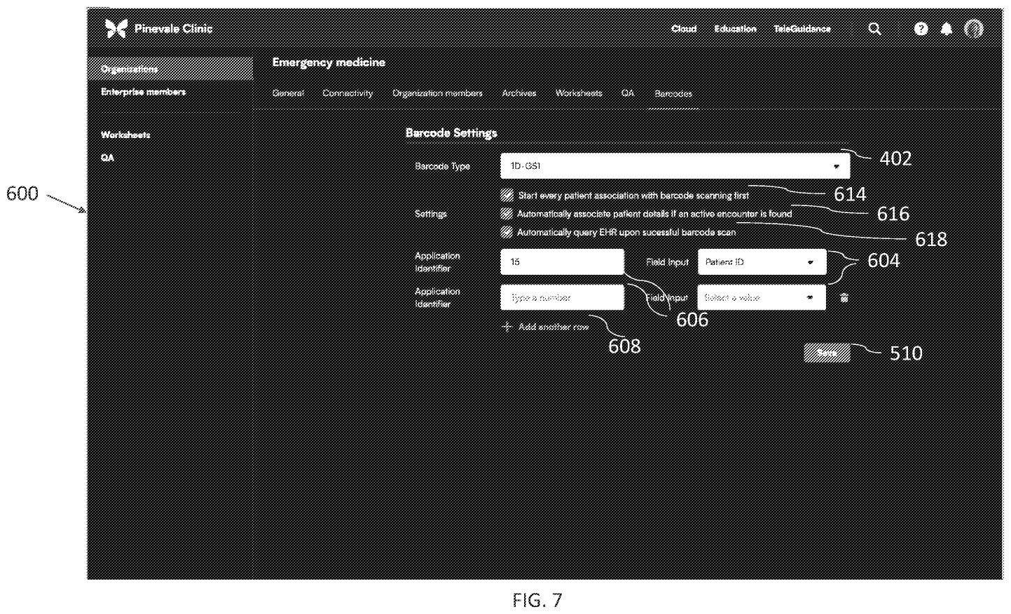

As another example, if a 1D GS1 barcode type is selected at step 202, then the processing device may prompt the user for input of AIs and a data type associated with each of them. FIG. 6 illustrates an example GUI 600 that may be displayed by the processing device (e.g., in conjunction with step 204), in accordance with certain embodiments described herein. The GUI 600 displays barcode data processing settings specific for selection of a 1D GSI barcode type (as selected from the menu 402). The GUI 600 includes a field 606 in which the user may input an AI value (in the example of FIG. 6, there is one AI with value 15) and a menu 604 from which the user may select the type of data encoded by the corresponding AI. In the example GUI 600, the data type options are first name, last name, date of birth, gender/sex, patient ID, encounter ID, or blank (i.e., any data encoded by the corresponding AI in the barcode is not used). FIG. 7 illustrates another example of the GUI 600, in accordance with certain embodiments described herein. In particular, FIG. 7 illustrates how the “Add another row” option 608 may be used to configure additional AIs and corresponding field types.

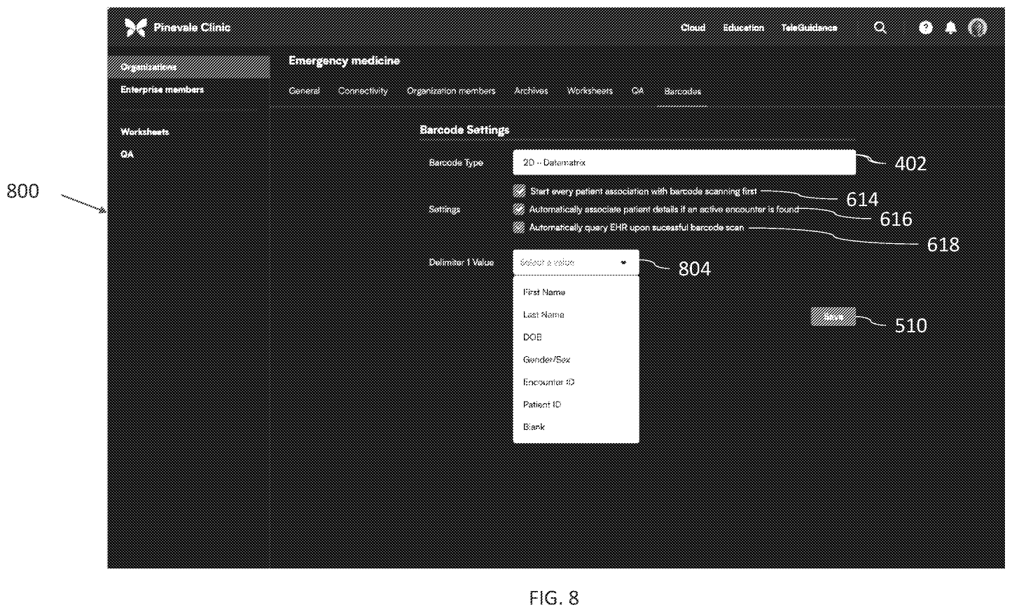

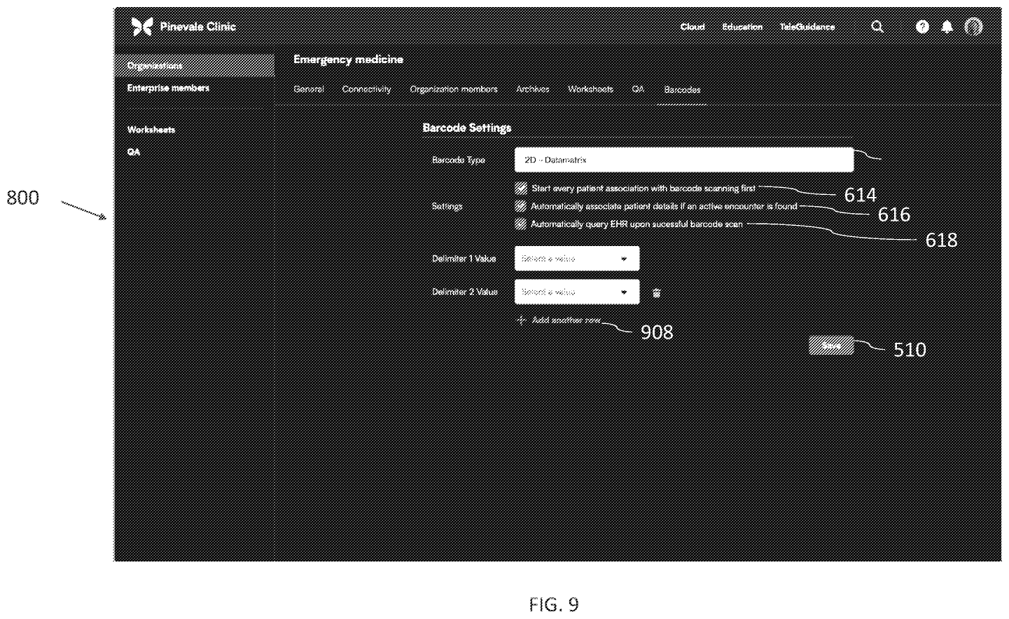

As another example, if a 2D barcode type is selected at step 202, then the processing device may prompt the user for selection of a certain number of delimiters and a data type associated with each of them. FIG. 8 illustrates an example GUI 800 that may be displayed by the processing device (e.g., in conjunction with step 204), in accordance with certain embodiments described herein. The GUI 800 displays barcode data processing settings specific for selection of a 2D barcode type (as selected from the menu 402). The GUI 800 includes a menu 804 from which the user may select the type of data encoded by a corresponding delimiter (in the example of FIG. 8, delimiter 1). In the example GUI 800, the data type options are first name, last name, date of birth, gender/sex, patient ID, encounter ID, or blank (i.e., any data encoded by the corresponding delimiter in the barcode is not used). FIG. 9 illustrates another example of the GUI 800, in accordance with certain embodiments described herein. In particular, FIG. 9 illustrates how the “Add another row” option 908 may be used to add additional delimiters and corresponding field types. The process 200 proceeds to step 206.

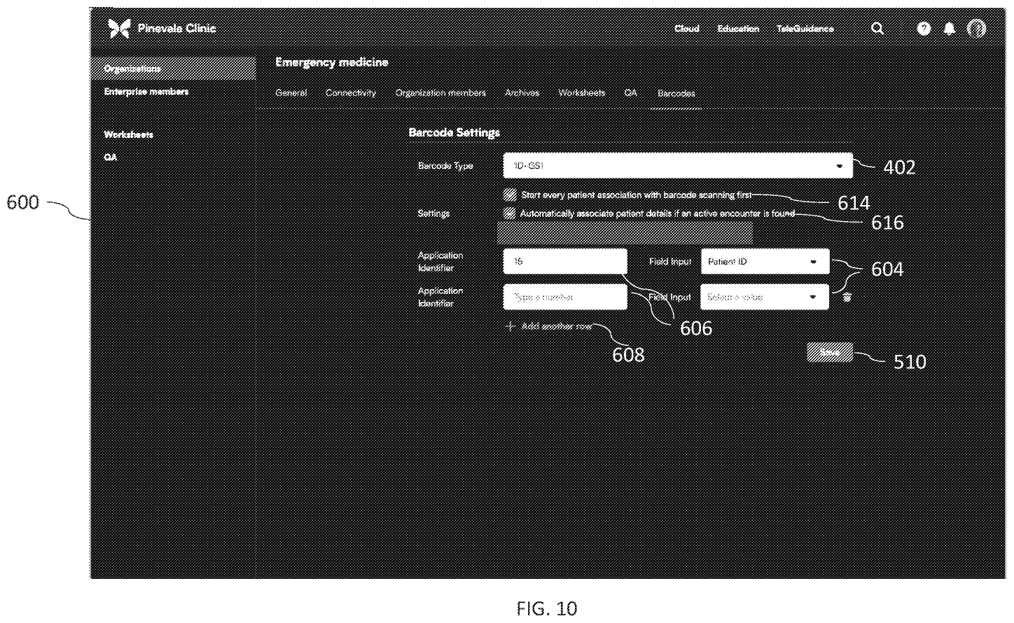

At step 206, the processing device determines whether automatic electronic health records (EHR) querying should be presented as an option. In some embodiments, the processing device may determine that EHR querying should be presented as an option based on querying using the healthcare interoperable standard FHIR® (Fast Healthcare Interoperability Resources) being enabled. Whether FHIR® querying is enabled may be a setting in the healthcare department's ultrasound software (i.e., the internet-based software being used on the processing device to select barcode settings). For a healthcare department using FHIR®, the data extracted from a barcode may be used to query the department's EHR to validate the extracted data. If EHR querying should be presented as an option, the process 200 proceeds to step 208. If EHR querying should not be presented as an option, the process 200 may terminate. FIG. 6 illustrates an example of the GUI 600 when EHR querying is presented as an option, namely the option 618. FIG. 10 illustrates another example of the GUI 600, in accordance with certain embodiments described herein. In particular, FIG. 10 illustrates an example when EHR querying is not presented as an option, as the option 618 is absent. It should be appreciated that the option 618 may be similarly displayed or not displayed in the GUIs 500 and 800.

At step 208, the processing device prompts for and receives a selection of whether to automatically perform an EHR query after an ultrasound scan. For example, the user may select or not select the option 618 from the GUIs 500, 600, or 800. In some embodiments, steps 206 and 208 may occur before or concurrently with either steps 202 or 204.

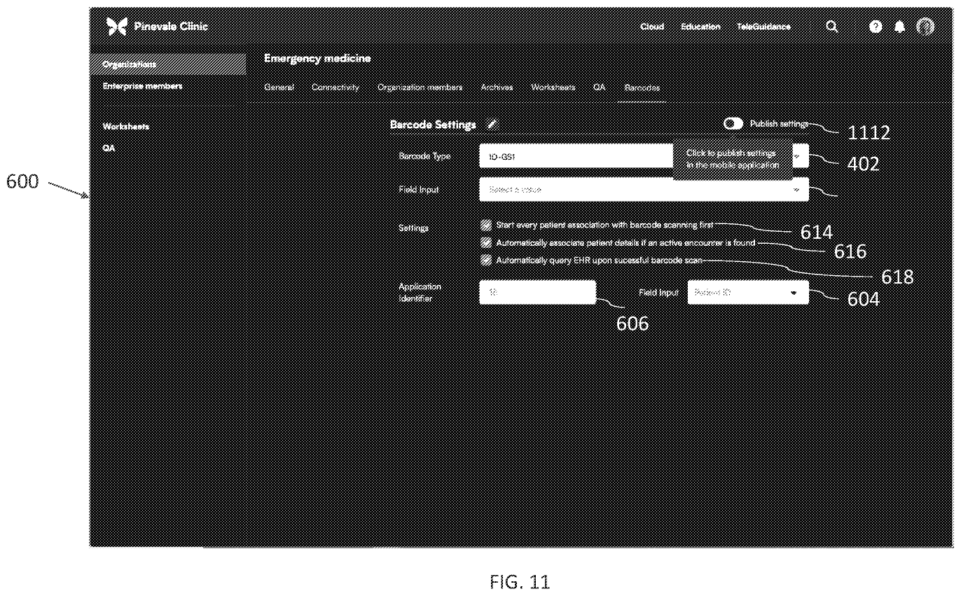

The barcode scanning settings may include the barcode type received at step 202, the configuration of how to process barcode data received at step 204, and (depending on the result of the determination at step 206) whether to automatically perform an EHR query after an ultrasound scan as received at step 208. Once selected in the cloud-based environment accessed by the processing device, the barcode settings can be downloaded from the cloud by mobile devices that are running the ultrasound application under an account linked to the department. FIG. 11 illustrates another example of the GUI 600, in accordance with certain embodiments described herein. In particular, FIG. 11 illustrates that the “Publish settings” option 1112 may be displayed once the “Save” option 510 has been selected (e.g., from any of the GUIs 500, 600, or 800). Upon selection of the “Publish settings” option 1112, the barcode settings selected using the GUI may be available on the cloud for download. It should be appreciated that the “Publish settings” option 1112 may be similarly displayed in the GUIs 500 and 800.

In some embodiments, the process 200 may be performed by a mobile device (e.g., the mobile device 104, which may be the same mobile device that performs the process 300 described below).

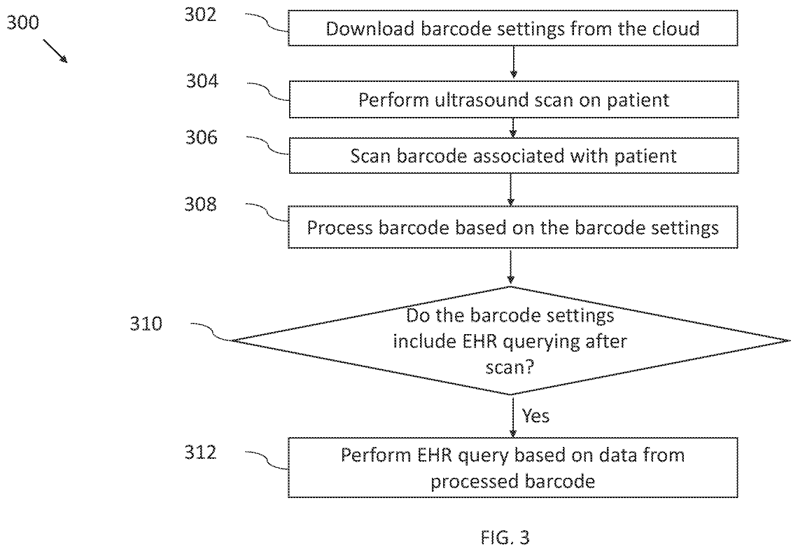

FIG. 3 illustrates a process 300 for scanning a patient barcode using an ultrasound application (e.g., the ultrasound application 114) running on a mobile device (e.g., the mobile device 104), in accordance with certain embodiments described herein.

At step 302, the mobile device downloads barcode settings from the cloud. The barcode settings may be those settings selected in the cloud in accordance with the process 200, namely the barcode type, the configuration of how to process barcode data, and whether to automatically perform an EHR query after an ultrasound scan. These barcode settings may be associated with a particular department. When a mobile device is running the ultrasound application under an account linked to a particular department, the ultrasound application may check whether any new department-specific settings (such as barcode settings) are available from the cloud, and if so, download them and configure the ultrasound application accordingly. The process 300 proceeds to step 304.

At step 304, the mobile device performs an ultrasound scan on a patient (e.g., the patient 112), in conjunction with an ultrasound device (e.g., the ultrasound device 102). For example, the ultrasound application on the mobile device may have a GUI that enables the user to configure the ultrasound device for a particular type of ultrasound scan, receive ultrasound data from the ultrasound device, display ultrasound images in the GUI, and upload ultrasound images to the cloud. The process 300 proceeds to step 306.

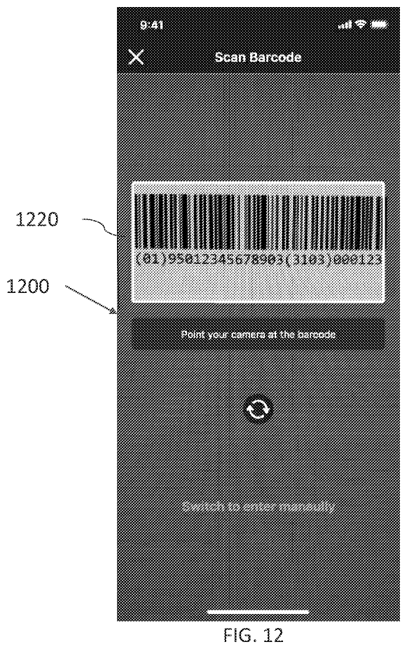

At step 306, the mobile device scans a barcode (e.g., the barcode 110) associated with the patient. The barcode may be, for example, printed on a bracelet worn by the patient. The mobile device may scan the barcode by capturing an image of the barcode using its camera. FIG. 12 illustrates an example GUI 1200 that may be displayed by the mobile device (e.g., in conjunction with step 306), in accordance with certain embodiments described herein. The GUI 1200 includes an image 1220 from the camera of the mobile device which the operator may view in order to capture the barcode. The process 300 proceeds to step 308.

At step 308, the mobile device processes the barcode (i.e., scanned at step 306) based on the barcode settings (i.e., the settings downloaded at step 302). For example, assume the barcode settings define the barcode to be a 1D non-GSI barcode type, and the settings define the data encoded by the barcode to be associated with a patient ID. At step 308, the mobile device would extract the data from the barcode and save it as the patient ID. As another example, assume the barcode settings define the barcode to be a 1D GSI barcode type, and the settings are those in Table 1. At step 308, the mobile device would extract multiple pieces of data from the barcode, determine from the barcode which data is associated with the AI 01 and which data is associated with the AI 3103, set the data associated with the AI 01 as the patient ID, and set the data associated with the AI 3103 as the encounter ID. As another example, assume the barcode settings define the barcode to be a 2D barcode type, and the settings are those in Table 2. At step 308, the mobile device would extract multiple pieces of data from the barcode, set the data associated with the first delimiter as the last name, set the data associated with the second delimiter as the first name, set the data associated with the third delimiter as the date of birth, set the data associated with the fourth delimiter as the gender/sex, set the data associated with the sixth delimiter as the encounter ID, and the data associated with the seventh delimiter as the patient ID. Any data associated with the sixth delimiter would not be used, as it is not associated with an information type. The process 300 proceeds to step 310.

At step 310, the mobile device determines whether the barcode settings include a setting to perform EHR querying after an ultrasound scan based on data from the processed barcode. If the barcode settings do include a setting to perform EHR querying, the process 300 proceeds to step 312.

At step 312, the mobile device performs an EHR query based on data from the processed barcode (i.e., processed at step 306). By querying the department's EHR, the data from the processed barcode may be validated against data in the EHR. For example, consider that the barcode contains a patient ID. The mobile device may query the department's EHR for any active encounters associated with the patient ID. As another example, consider that the barcode contains an encounter ID. The mobile device may query the department's EHR for any active encounters with this ID. As another example, consider that the barcode contains both a patient ID and an encounter ID. The mobile device may query the department's EHR for any active encounters with this encounter ID and/or associated with patient ID. As another example, consider that the barcode contains neither a patient ID nor an encounter ID. The mobile device may then query the EHR for any active encounters.

In some embodiments, the mobile device may return a list of matching encounters from the EHR query and prompt the user to select one. Once the user selects an encounter from the list, the mobile device may associate the ultrasound scan with patient information from the selected encounter. In some embodiments, if there is only one matching encounter from the EHR query, the mobile device may automatically associate the ultrasound scan with patient information from this encounter, without the user needing to select the encounter. In some embodiments, during barcode configuration (e.g., the process 200), the processing device may prompt for and receive a selection (e.g., the setting 616 in the illustrated GUIs) as to whether the mobile device should automatically associate the scan with patient information if there is only one matching encounter from the EHR query. If no matching encounters are found, the user may be prompted to select an encounter from a list of active encounters, and populate the patient information with information from the selected encounter. In some embodiments, the user may need to confirm that selected patient information should be associated with the scan before the association is actually performed.

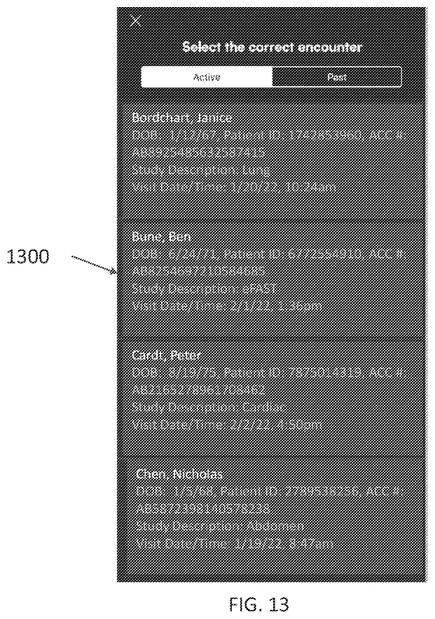

FIG. 13 illustrates an example GUI 1300 that may be displayed by the mobile device (e.g., in conjunction with step 312), in accordance with certain embodiments described herein. In particular, the GUI 1300 illustrates an example list of encounters from which the user may select patient information in any of the scenarios discussed above.

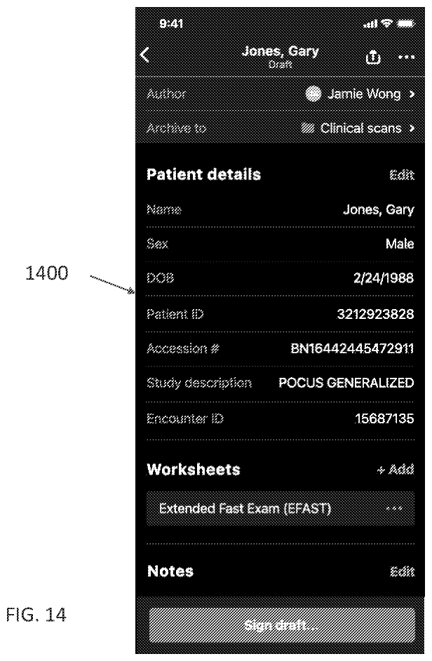

FIG. 14 illustrates an example GUI 1400 that may be displayed by the mobile device (e.g., in conjunction with step 312), in accordance with certain embodiments described herein. In particular, the GUI 1400 illustrates example patient information be associated with a scan.

Thus, if EHR querying is performed, patient information associated with a scan may come from the department's EHR based on a query of extracted barcode data. If EHR querying is not performed, patient information associated with a scan may come directly from the extracted barcode data, without validation from the department's EHR. The mobile device may further provide the user with the option to manually enter other information not contained in the barcode. In any case, once patient information has been associated with an ultrasound scan, the scan (i.e., ultrasound images, ultrasound cines, worksheets completed based on the images/cines, etc.) may be uploaded to the cloud along with the patient information.

In some embodiments, during barcode configuration (e.g., the process 200), the processing device may prompt for and receive a selection (e.g., the setting 614 in the illustrated GUIs) as to whether the mobile device should automatically start every patient association with barcode scanning (e.g., rather than prompting for a selection of whether to scan a barcode during every patient association step).

In some embodiments, steps 306, 308, 310, and/or 312 may occur before step 304. In other words, all are certain portions of the barcode scanning, processing, and EHR querying may be performed before the ultrasound scan. In some embodiments, step 302 may occur after step 304. In other words, barcode settings may be downloaded after the ultrasound scan is performed.

As described above, department-specific barcode settings downloaded from the cloud at step 302 of the process 300 may have been selected in accordance with the process 200. Indeed, a process may include the process 200 followed by the process 300. For example, a process may proceed from step 206 or 208 to step 302.

In some embodiments, EHR querying may always be presented as an option. Thus, step 206 may be absent in the process 200. In some embodiments, EHR querying may always be performed. Thus, steps 206 and 208 may be absent in the process 200 and step 310 may be absent in the process 300. In such embodiments, the barcode settings configured in the process 200 and downloaded at step 302 of the process 300 may include the barcode type and the configuration for how to process barcode data, but need not include a selection of whether to perform EHR querying after an ultrasound scan. In some embodiments, EHR querying may never be performed. Thus, steps 206 and 208 may be absent in the process 200 and steps 310 and 312 may be absent in the process 300. Again, in such embodiments, the barcode settings configured in the process 200 and downloaded at step 302 of the process 300 may include the barcode type and the configuration for how to process barcode data, but need not include a selection of whether to perform EHR querying after an ultrasound scan.

It should be appreciated that the term “department” as used herein in the specification and in the claims, should be understood to mean a department or any other type of medical group, such as a medical practice, clinic, hospital, or medical school (as non-limiting examples).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within +20% of a target value in some embodiments, within +10% of a target value in some embodiments, within +5% of a target value in some embodiments, and yet within +2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound system, comprising:
an ultrasound device;
a mobile device in operative communication with the ultrasound device and running an ultrasound application;
a processing device; and
a cloud comprising one or more servers, wherein the ultrasound device and the processing device are in communication with the cloud;
wherein the processing is configured to:
prompt for and receive a selection of a barcode type; and
prompt for and receive configuration of how to process barcode data, wherein the prompt is based on the selected barcode type; and
wherein the mobile device is configured to:
download, from the cloud, barcode settings comprising the barcode type and the configuration of how to process the barcode data;
perform the ultrasound scan on a patient in conjunction with the ultrasound device;
scan a barcode associated with the patient;
process the barcode data based on the downloaded barcode settings; and
perform an electronic health record (EHR) query based on data from the processed barcode.

2. The ultrasound system of claim 1, wherein the processing device is configured, when prompting for and receiving the selection of the barcode type, to prompt for and receive a selection of a 1D non-GSI barcode, a 1D GS1 barcode, or a 2D barcode.

3. The ultrasound system of claim 1, wherein the processing device is configured, when prompting for and receiving the configuration of how to process the barcode data, to prompt for a selection of a data type encoded by the barcode when the selected barcode type is a 1D non-GSI barcode.

4. The ultrasound system of claim 3, wherein the data type comprises patient ID or an encounter ID.

5. The ultrasound system of claim 1, wherein the processing device is configured, when prompting for and receiving the configuration of how to process the barcode data, to prompt for input of application identifiers and a data type associated with each of the application identifiers when the selected barcode type is a 1D GSI barcode.

6. The ultrasound system of claim 5, wherein the data type comprises a first name, last name, date of birth, gender/sex, patient ID, encounter ID, or blank.

7. The ultrasound system of claim 1, wherein the processing device is configured, when prompting for and receiving the configuration of how to process the barcode data, to prompt for selection of a certain number of delimiters and a data type associated with each of the delimiters.

8. The ultrasound system of claim 7, wherein the data type comprises a first name, last name, date of birth, gender/sex, patient ID, encounter ID, or blank.

9. The ultrasound system of claim 1, wherein the processing device is further configured to determine that automatic EHR querying should be presented as an option.

10. The ultrasound system of claim 9, wherein the processing device is configured to determine that automatic EHR querying should be presented as an option based on querying using Fast Healthcare Interoperability Resources being enabled.

11. The ultrasound system of claim 9, wherein the processing device is further configured to prompt for and receive a selection of whether to automatically perform the EHR query based on the data from the processed barcode after the ultrasound scan.

12. The ultrasound system of claim 11, wherein the barcode settings further comprise whether to automatically perform the EHR query based on the data from the processed barcode after the ultrasound scan.

13. The ultrasound system of claim 1, wherein the barcode settings are associated with a particular department, and the mobile device is further configured, when downloading the barcode settings from the cloud, to check whether new department-specific settings are available from the cloud when running the ultrasound application under an account linked to the particular department.

14. The ultrasound system of claim 1, wherein:
the mobile device is configured, when scanning the barcode associated with the patient, to scan a barcode printed on a bracelet worn by the patient; and
the mobile device comprises a camera and is configured, when scanning the barcode associated with the patient, to capture an image of the barcode using the camera.

15. The ultrasound system of claim 1, wherein the mobile device is configured, when performing the EHR query based on the data from the processed barcode, to query the EHR for active encounters associated with a patient ID when the barcode contains the patient ID.

16. The ultrasound system of claim 1, wherein the mobile device is configured, when performing the EHR query based on the data from the processed barcode, to query the EHR for active encounters associated with an encounter ID when the barcode contains the encounter ID.

17. The ultrasound system of claim 1, wherein the mobile device is further configured to:
return a list of matching encounters from the EHR query;
prompt for selection of one of the matching encounters; and
associate the ultrasound scan with patient information from the selected encounter.

18. The ultrasound system of claim 1, wherein the mobile device is further configured, when there is only one matching encounter from the EHR query, to automatically associate the ultrasound scan with patient information from the matching encounter.

19. The ultrasound system of claim 1, wherein the processing device is further configured to prompt for and receive a selection as to whether the mobile device should automatically associate the ultrasound scan with patient information if there is only one matching encounter from the EHR query.

20. The ultrasound system of claim 1, wherein the processing device is further configured to prompt for and receive a selection as to whether the mobile device should automatically start every patient association with barcode scanning.
